(19) United States
(12) Patent Application Publication
(10) Pub. No.: US 2024/0301393 A1
(43) Pub. Date: Sep. 12, 2024

(54) METHOD AND SYSTEM FOR EXTRACTION, PURIFICATION, ANALYSIS AND/OR DETECTION OF NUCLEIC ACID MOLECULES

(71) Applicant: NATIONAL UNIVERSITY OF SINGAPORE, Singapore (SG)

(72) Inventors: Chung-Pei OU, Singapore (SG); Yong Kiat LIM, Singapore (SG)

(21) Appl. No.: 18/570,994

(22) PCT Filed: Jun. 16, 2022
(86) PCT No.: PCT/SG2022/050417

§ 371 Date: Dec. 15, 2023

(30) Foreign Application Priority Data
Jun. 18, 2021 (SG) 10202106616X

(51) Int. Cl. C12N 15/10; B01L 7/00

(52) U.S. Cl. CPC C12N 15/1006; B01L 7/52; B01L 2200/0647

(57) ABSTRACT

The invention relates to various components that make up a system for extracting, purification, analysis and/or detection of nucleic acid molecules. A first component comprises a nucleic acid capturing device adapted for dipping into a liquid wherein a portion of the device contacting the liquid comprises a nucleic acid binding agent. The nucleic acid binding agent may comprise silica (SiO₂). The invention also includes a method for producing said nucleic acid capturing device. A second component comprises a grinding device comprising a mortar and pestle with the mortar comprising an upper portion and a base grinding portion. The grinding device enables efficient grinding in the base grinding portion and facilitates access to the addition of materials (e.g. fluids to the base grinding portion) when in use. A third component is an amplification device (e.g. a heating device for isothermal amplification).

### FIELD OF THE INVENTION

The present invention relates to extraction, purification, analysis and/or detection of nucleic acid molecules. In particular, the present invention relates to purification and analysis of nucleic acid molecules, for example to detect/identify microorganisms. More in particular, the present invention relates to molecular diagnostics, genetic testing, breed selection testing, GMO testing, pathogen detection, genotyping, mutation detection for veterinary (specifically aquaculture) usage for detection, prescription or genetic treatment.

### BACKGROUND OF THE INVENTION

Traditional methods of detecting nucleic acids, which may occur in minute quantities in collected samples, involve multiple devices and steps to process a sample, amplify targets and detect the amplification process. The steps to process a sample are to extract the nucleic acid, DNA and/or RNA from the sample, where others components can cause interference during the amplification. The extraction process is usually performed by a skilled technician in a laboratory using a nucleic acid extraction kit such as Qiagen DNA mini prep and requires specialised equipment, for example precision pipettes, and centrifuge.

The amplification process and workflows are well established with different methods being established for the conduct of different assays. After amplification, there are well established methods to visualize the results of the amplification process. Such methods include attaching a probe to the amplification process. Such probes include fluorescence tag in Taqman detection oligos, or other fluorescence chemical dyes that produce discrete optical spectrum change during the amplification of nucleic acid. Typically, the discrete change is proportional to the amount of amplification product. Applications of such includes quantitative PCR. Other methods of visualising include agarose gel electrophoresis, or pH indicators to indicate the hydrogen ions which are the by-products of the amplification and thus proportional to the amount of amplification.

This has led to a lengthy, expensive process of sending samples to be tested occasionally at a fully equipped laboratory, which is both expensive and time-consuming. Farmers face a wait of between 24-48 hours from obtaining the samples to get a confirmatory diagnostic result.

It is therefore desirable to simplify extraction, purification, analysis and/or of nucleic acid molecules.

### SUMMARY OF THE INVENTION

According to a first aspect, the present invention relates to a nucleic acid capturing device adapted for dipping into a liquid wherein a portion of the device contacting the liquid comprises a nucleic acid binding agent.

According to another aspect, the present invention relates to a method for capturing nucleic acid from a liquid comprising:
(i) providing a nucleic acid capturing device adapted for dipping into the liquid wherein a portion of the device contacting the liquid comprises a nucleic acid binding agent; and
(ii) contacting said portion of the device with the liquid.

The present invention also includes a method for coating a portion of a device for capturing nucleic acid comprising:
(i) contacting a portion of the device with a solution of tetraethyloxysilane, tetramethoxysilane and/or vinyltriethoxysilane in ethanol for a time period;
(ii) adding multiple aliquots of an acid or a base to the solution periodically;
(iii) removing the portion of the device from the solution of (ii); and
(iv) optionally drying the device.

The invention also includes a grinding device comprising:
(i) a mortar comprising a base grinding portion and an upper portion wherein a first opening at the top of the upper portion is larger than a second opening to the base grinding portion; wherein the second opening delineates the end of the top portion and opens to the base grinding portion; and
(ii) a pestle adapted to fit into the base grinding portion for grinding a material in the base grinding portion.

The invention further includes a grinding method comprising grinding a material in a grinding device comprising:
(i) a mortar comprising a base grinding portion and an upper portion wherein a first opening at the top of the upper portion is larger than a second opening at the base of the upper portion; wherein the second opening delineates the end of the top portion and opens to the base grinding portion; and
(ii) a pestle adapted to fit into the base grinding portion for grinding a material in the base grinding portion.

According to a further aspect, the invention relates to a system for purification, analysis and/or detection of nucleic acid molecules comprising:
(i) the nucleic acid capturing device as described herein; and
(ii) a heating device.

The present invention also relates to a system for extraction, purification, analysis and/or detection of nucleic acid molecules; comprising:
(i) the grinding device as described herein; and
(ii) the nucleic acid capturing device as described herein.

The present invention further includes a method for extraction, purification, analysis and/or detection of nucleic acid molecules comprising:
(i) contacting a nucleic acid capturing device as described herein with a liquid (e.g. a binding buffer) to capture nucleic acid molecules;
(ii) optionally washing the portion of the device comprising a nucleic acid binding agent;
(iii) releasing the captured nucleic acid molecules from the device into a solution.

The present invention further includes a method for extraction, purification, analysis and/or detection of nucleic acid molecules comprising:
(i) contacting a device as described herein with a liquid (e.g. a binding buffer) to capture nucleic acid molecules;
(ii) optionally washing the portion of the device comprising a nucleic acid binding agent;
(iii) releasing the captured nucleic acid molecules from the device into a solution; and
(iv) analysing and/or detecting the nucleic acid molecules in the solution.

### BRIEF DESCRIPTION OF THE FIGURES

FIG. 1 shows an embodiment of the grinding device. (A) shows the top view of the mortar; (B) shows the side view of the pestle (C) shows the pestle.

FIG. 2 shows two embodiments of the nucleic acid collecting device comprising a nucleic acid binding portion at one end. (A) shows the nucleic acid collecting device comprising a nucleic acid binding agent at one end. (B) shows a nucleic acid collecting device comprising a porous structure.

FIG. 3 shows coated sticks (si-sticks).

### DEFINITIONS

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “functionalized,” when referring to a surface, means a surface which has been modified by a material.

### DETAILED DESCRIPTION OF THE INVENTION

According to a first aspect, the present invention relates to a nucleic acid capturing device adapted for dipping into a liquid wherein a portion of the device contacting the liquid comprises a nucleic acid binding agent.

According to another aspect, the present invention relates to a method for capturing nucleic acid from a liquid comprising:
(i) providing a nucleic acid capturing device adapted for dipping into the liquid wherein a portion of the device contacting the liquid comprises a nucleic acid binding agent; and
(ii) contacting said portion of the device with the liquid.

It will be appreciated that the device according to the first aspect of the invention may be used for said method.

For the invention as described herein, the portion of the device contacting the liquid comprises a surface functionalised with a nucleic acid binding agent. Any suitable nucleic acid binding agent is contemplated. In particular, the nucleic acid binding agent may comprise silica (SiO₂). As one example of the invention described herein, the portion of the device contacting the liquid may comprise a porous structure comprising the nucleic acid binding agent.

The present invention also includes a method for coating a portion of a device for capturing nucleic acid comprising:
(i) contacting a portion of the device with a solution of tetraethyloxysilane, tetramethoxysilane and/or vinyltriethoxysilane in ethanol for a time period;
(ii) adding multiple aliquots of an acid or a base to the solution periodically;
(iii) removing the portion of the device from the solution of (ii); and
(iv) optionally drying the device.

It will be appreciated that drying the device may be performed after the coating process (with all repeats) is completed. The device should be dry before using to capture nucleic acid molecules.

It will be appreciated that (i) to (iv) may be repeated at least once. For example, (i) to (iv) may be repeated once or twice.

For example, tetraethyloxysilane may be used for each (i). It will be appreciated that the amount of tetraethyloxysilane for each (i) for the first instance and each repeat can be the same or different. It will also be appreciated that the time period for each (i) for the first instance and each repeat may different or the same.

### For Example

Each (i) independently may comprise contacting a portion of the device with 10-90% v/v of tetraethyloxysilane.

Each (i) independently may comprise contacting a portion of the device with approximately 35% v/v of tetraethyloxysilane.

Each (i) for the first instance and the first repeat may comprise contacting a portion of the device with 10-90% v/v of tetraethyloxysilane.

Each (i) for the first instance and the first repeat may comprise contacting a portion of the device with approximately 35% v/v of tetraethyloxysilane.

Independently, (i) for the second repeat may comprise contacting a portion of the device with 10-90% v/v tetraethyloxysilane.

Independently, (i) for the second repeat may comprise contacting a portion of the device with 25% v/v tetraethyloxysilane.

The time period for each (i) independently, may be or is approximately 20 minutes.

The time period for each (i) for the first instance and the first repeat is approximately 20 minutes.

The time period for (i) for the second repeat independently is approximately 30 minutes.

The acid in (ii) includes but is not limited to citric acid or hydrogen chloride. The base in (ii) includes but is not limited to ammonia or sodium chloride

It will also be appreciated that the amount of base or acid for each (ii) for the first instance and each repeat may be different or the same.

Using NaOH for the purpose of illustrating the following examples:

The sodium chloride solution for each (ii) independently, may comprise or comprises approximately 17% w/v NaOH.

The sodium chloride solution for each (ii) for the first instance and the first repeat comprises approximately 17% w/v NaOH,

The sodium chloride solution for (ii) for the second repeat independently, comprises approximately 17% w/v NaOH,

It will be appreciated for each (ii) independently, the base or acid may be added at the same periodic intervals or different periodic intervals.

Using NaOH for the purpose of illustrating the examples:

Each (ii) independently, may comprise or comprises adding NaOH at approximately 5 minute intervals.

Each (ii) for the first instance and the first repeat comprises adding NaOH at approximately 5 minute intervals.

For the second repeat independently, (ii) may comprise adding NaOH at approximately 9 minute intervals.

The method for coating a portion of a device for capturing nucleic acid as described herein further comprises optionally sonicating a portion of the device in said solution after each (ii).

The invention also includes a grinding device comprising:
(i) a mortar comprising a base grinding portion and an upper portion wherein a first opening at the top of the upper portion is larger than a second opening to the base grinding portion; wherein the second opening delineates the end of the top portion and opens to the base grinding portion; and
(ii) a pestle adapted to fit into the base grinding portion for grinding a material in the base grinding portion.

In particular, the bottom of the base grinding portion has substantially the same dimension as the second opening. In one embodiment, the wall of the base grinding portion extends substantially perpendicularly from its bottom to the second opening of the top portion.

In another embodiment, the base grinding portion of the grinding device may further comprise a buffer. In a further embodiment, the first opening of the grinding device may be sealed with a removable seal.

It will be appreciated that the grinding device as described may be used for grinding a material.

Accordingly, the invention further includes a grinding method comprising grinding a material in a grinding device comprising:
(i) a mortar comprising a base grinding portion and an upper portion wherein a first opening at the top of the upper portion is larger than a second opening at the base of the upper portion; wherein the second opening delineates the end of the top portion and opens to the base grinding portion; and
(ii) a pestle adapted to fit into the base grinding portion for grinding a material in the base grinding portion.

According to a further aspect, the invention relates to a system for purification, analysis and/or detection of nucleic acid molecules comprising:
(i) the nucleic acid capturing device as described herein; and
(ii) a heating device.

The present invention also relates to a system for extraction, purification, analysis and/or detection of nucleic acid molecules; comprising:
(i) the grinding device as described herein; and
(ii) the nucleic acid capturing device as described herein.

The system for extraction, purification, analysis and/or detection of nucleic acid molecules may further comprise a polymerase chain reaction (PCR) machine or a heating device. The PCR or heating device may be adapted to carry out nucleic acid amplification in a reaction container. This system may further comprise a device for analysis, detection and/or identification of a target nucleic acid molecule. This system may also further comprise at least one computer processor. The computer processor may be adapted to send a signal to stop and/or prevent access to the PCR machine and/or the heating device. The computer processor(s) may communicate with other computer processor(s) in the system and/or with one or more other computer systems.

The present invention further includes a method for extraction, purification, analysis and/or detection of nucleic acid molecules comprising:
(i) contacting a nucleic acid capturing device as described herein with a liquid (e.g. a binding buffer) to capture nucleic acid molecules;
(ii) optionally washing the portion of the device comprising a nucleic acid binding agent;
(iii) releasing the captured nucleic acid molecules from the device into a solution.

The method may further comprise (iv) amplifying the nucleic acid molecules. Any nucleic acid amplification may be used including but not limited to amplifying the nucleic acid molecules with PCR, nucleic acid sequence based amplification (NASBA), recombinase polymerase amplification (RPA), nicking enzyme amplification reaction (NEAR), rolling-circle amplification (RCA), helicase-dependent amplification (HDA), multiple displacement amplification (MDA), CRISPR/Cas based isothermal amplification, or isothermal amplification (e.g. mediated amplification (LAMP)).

For example, (iv) comprises amplifying the nucleic acid molecules comprises amplifying the nucleic acid molecules with PCR in a PCR machine. As another example, (iv) comprises amplifying the nucleic acid molecules comprises amplifying the nucleic acid molecules with isothermal amplification (e.g. LAMP) in a heating device.

The method for extraction, purification, analysis and/or detection of nucleic acid molecules further comprises at least one computer processor sending a signal to sop the PCr machine or heating device, allow access of the PCR machine or heating device to an authorised person and/or prevent access to the PCT machine or heating device.

The method for extraction, purification, analysis and/or detection of nucleic acid molecules further comprises analysing and/or detecting the nucleic acid molecules after step (iii) or (iv).

Prior to step (i), a grinding device may be used to grind a material. This is to release nucleic acid molecules for extraction, purification, analysis and/or detection. It will be appreciated that the grinding device as described herein may be used.

The invention comprises the following aspects: the kit for breaking up cell(s) and/or tissue(s), the nucleic acid extraction kit (collectively thereafter referred to as the sample preparation kit) and/or the amplification device (e.g. heating device).

Having now generally described the invention, the same will be more readily understood through reference to the following examples which are provided by way of illustration, and are not intended to be limiting of the present invention.

### EXAMPLES

### Example 1: Sample Preparation

Tissue samples are obtained from prawns. These tissue samples can include but are not limited to prawn muscle tissue, prawn shell tissue, prawn haemolymph, prawn hepatopancreas, whole prawn zoea, whole prawn larvae, whole prawn post-larvae. The sample size can be from quantities such as 0.1 g to 5 g.

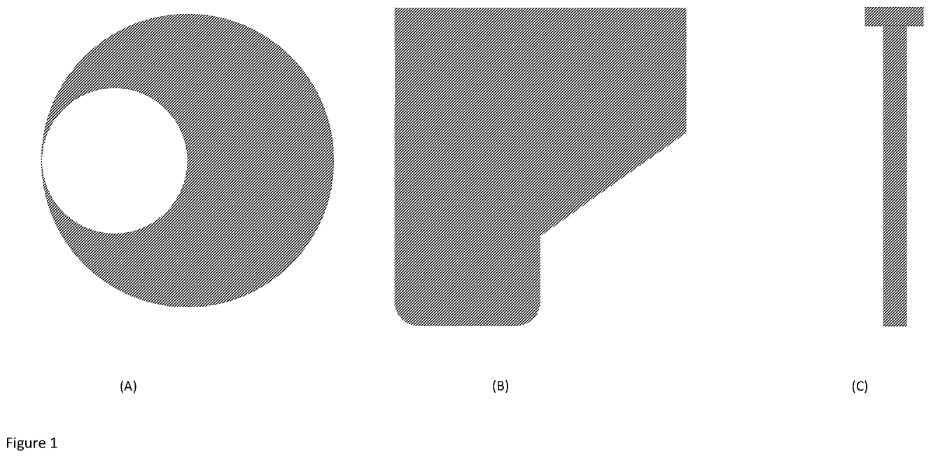

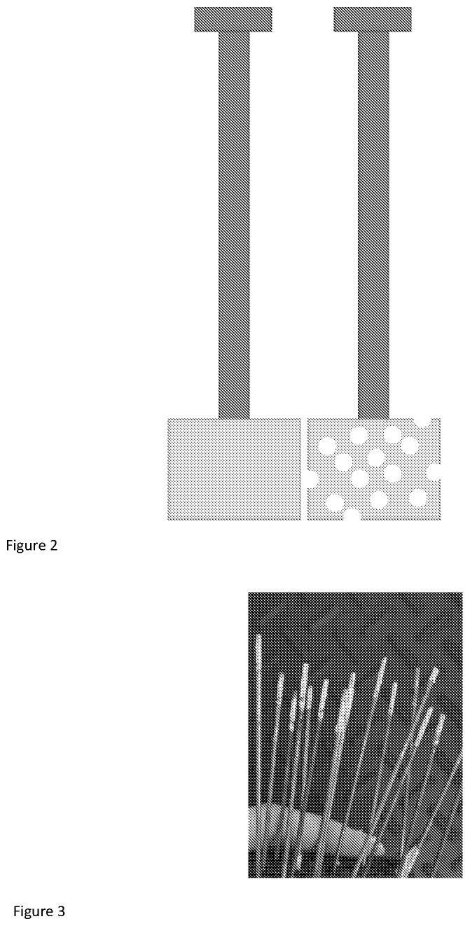

These samples will then be broken up to release the nucleic acid from the cell(s) and/or tissue(s). The kit for breaking up the cell(s) and/or tissue(s) is comprised of at least the mortar-grinder set and reagents. The mortar-grinder set comprises of a mortar as well as a grinder. In an exemplary embodiment, the mortar comprises of 2 portions which are aligned on one side (refer to FIG. 1). The mortar comprises an upper portion and a base grinding portion, with a first opening at the top of the upper portion that is larger than a second opening at the base of the upper portion; wherein the second opening delineates the end of the top portion and opens to the base grinding portion. The mortar can fit at least about 30 prawn pleopods. For this embodiment, the size of the mortar and grinder is important as 30 prawn pleopods per batch is in line with a 10% infection prevalence. Alternatively, the user can perform two preparations (60 pleopods) to achieve a 5% infection prevalence. A grinder, of a suitable size for the mortar is then used to crush the prawn pleopods. The grinder may be around the same size as the second opening or 10% to 90% of the size of the second opening. Reagents can be added on the slope of the mortar, along with deionized water to hydrate the reagents.

It will be appreciated that the size of the mortar and grinder device can be adapted for the cell(s)/tissue(s) that are to be homogenised accordingly.

In one exemplified embodiment, the nucleic acid extraction kit includes a nucleic acid capturing device comprising an elongated member, which carries a nucleic acid binding area at the end of the elongated member.

The nucleic acid binding area may comprise a nucleic acid binding material, such as silica (SiO₂) covering the surface of the tip end. The design is such that very little elution solution (e.g. water or TE buffer) is required to elute nucleic acid from the stick and in addition, less than 10% of residual solution remains trapped in the elongated member. In such a way, all the eluted solution can be used for nucleic acid analysis and detection in the amplification reaction. The elution yield can reach 90% or higher without laboratory equipment, such as centrifuges or vacuum pump. The elution yield is critical to the sensitivity of the nucleic acid detection, which is required at 10 copies to 1000 copies of pathogen per assay for example.

Alternatively, the surface of the tip end of the elongated member comprises a porous structure, such as a mesh or a multi-layer of mesh. The porous structure may comprise a nucleic acid binding agent. In an exemplary embodiment, the porous structure may comprise a silica membrane which provides a larger surface area to increase the binding capacity and recovery yield of nucleic acid. The dimension of the silica membrane is such that there is little residual solution trapped in the membrane during the elution.

### Example 2: Heating Device

The heating device comprises of a heating mechanism and a mobile/wifi enabled controller. The heating mechanism is able to heat samples up to 65° C. for 30 minutes. This heating mechanism can come in the forms of a water bath, heat block or any other mechanism which is able to convey heat evenly and consistently for a period of time. The controller is able to perform the following actions:
1. Control the heating mechanism to heat samples at a constant temperature for a period of time
2. able to read/transmit information to a back-end server via a mobile network/wifi network

Most importantly, the heating device has an algorithm to give an alert of the heightened risk of disease outbreak. The disease out-break alert is communicated to a remote site by wireless communication or/and through the display of the device. The remote site can be a cloud server or a standalone terminal of an authorised agent.

It is not rare that farmers may under report the outbreak for fearing of forced deposition of their stock or avoiding the intervention. To enforce the biosecurity, a two-factor method is invented to address the issue. First, once an alert is triggered by the device according to the algorithm, the device will be disabled to prevent future testing. This is to prevent the outbreak being underreported.

Each device has a unique identification number which can also be read as a bar code printed on the device. A one-time passcode can be obtained by an authorised agent or the farmer by registering the incident using the unique identification number with the service provider via internet or a phone call to the service provider. The one-time passcode can be used to unlock the device.

The algorithm for giving an alert is based on 95% confidence interval of the positive infection to disease outbreak. This involve repeating at least a second assay, if the first assay shows positive infection.

### Example 3: Workflow

1. Tissue samples are collected from a cohort of prawns ranging from 15-30 pieces, depending on the number of individuals growing in the area being sampled. These tissue samples can include but are not limited to prawn muscle tissue, prawn shell tissue, prawn haemolymph, prawn hepatopancreas, whole prawn zoea, whole prawn larvae, whole prawn post-larvae.
2. The samples are placed in the mortar. The reagents, e.g. lysozyme, and some water is added. The samples are then pounded using the grinder. This process continues for around 1 minute, until the samples are pulverised.
3. The mortar, containing the samples, is then placed into the heating device, which has been pre-heated. Incubation is carried out for 30 minutes at 50-55° C.
4. Upon completion of the incubation process, the packaging of the nucleic extraction kit is then removed. Binding buffer is added to the sample. The elongated member is inserted into the lysed sample, and is agitated in the liquid, akin to a stirring motion. This process goes on for 1 minute for example. The elongated member is then removed from the liquid in the mortar, with care taken not to remove solid debris.
5. Dip the tip of the elongated member in washing solution for 15 seconds. Dab dry with a clean piece of paper or fabric or absorbent material.
6. The captured nucleic acid is then transferred into solution in an amplification reaction chamber. Fill the amplification reaction chamber with water or buffer. Dip the tip of the elongated member into the amplification reaction chamber. Stir and agitate for 30 seconds to allow the nucleic acid to un-bind from the collector tip. Remove the collector tip from the reaction chamber. Add amplification reagents in the reaction chamber.
7. Place amplification reaction chamber into the device and heat for 30 minutes at 65° C. The device will read the results automatically; failing which, the results can be read by the naked eye.

### Example 4: Developing a Silicon Dioxide Coating Technique (Si-Stick)

A technique was developed to produce an even and resistant coating on a variety of materials. Materials such as plastic (polycarbonate, polylactic acid) and metals (iron, steel, aluminum) are able to be coated.

Ten sticks on a holder were suspended over a solution of 25 mL tetraethyloxysilane, 30 mL of 99% ethanol and 10-15 mL water, with 150 uL of 17% w/v sodium hydroxide solution being added every 5 minutes. After 20 minutes, the sticks were transferred to an ultrasonicator. The ultrasonicator was filled with a solution comprising of 15 mL of tetraethyloxysilane, 30 mL of 99% ethanol, and 10-15 mL water. The ultrasonicator was then turned on for 30 minutes of treatment, 100 ul of 17% w/v sodium hydroxide was added every 9 minutes. Upon completion after 30 minutes, the sticks are removed and placed in an oven for drying over 4 hours. The two-step approach of coating results in a surface that is mechanically strong and has a good adhesion of silica particle aggregates, which is critical for nucleic acid binding and extraction. The coated sticks are shown in FIG. 3.

It will be appreciated that to form a functional layer for nucleic acid extraction, a surface consists of silica (SiO₂) can be produced by hydrolysis and condensation of silica precursors, such as tetraethoxysilane (also known as tetraethyl orthosilicate, TEOS), tetramethoxysilane (also known as tetramethyl orthosilicate, TMOS) and vinyltriethoxysilane. The silica precursors can be used as a single or a mixture to achieve the desired adhesion to the underlying substrate, morphology, mechanical strength, vibrational resistant, surface area, roughness of the deposited silica surface on the nucleic acid capture device along with modifiers such as polyvinylpyrrolidone (PVP).

The formation of silica (Silicon dioxide) can be divided into two phases: hydrolysis of silica precursor and condensation of silicic acid from the hydrolysis. The presence of water will trigger the hydrolysis of the silica precursors, which is also modulated by the acidity and reactivity of the solution. The condensation step is influenced by the pH, reactivity, and the presence of nucleation sites. It will be appreciated that the hydrolysis can be modulated by using ammonia, sodium hydroxide, citrate acid, hydrogen chloride, to name a few.

### Example 5: Discussion

Genetic testing has been widely used in clinical application, aquaculture industry, pathogen epidemic surveillance. This genetic testing covers a range of technologies that involve detection & identification of nucleic acids from samples. Examples include DNA sequencing, real time polymerase chain reactions (RT-PCR), DNA microarray, loop mediated amplification (LAMP).

The present invention provides for means for preparing nucleic acid from a biological sample and a device for LAMP genetic testing, both to be conducted outside of labs in a farm-site environment by non-skilled personnel.

The present invention relates to an integrated system, consumables kit and workflow that is suitable for deployment in rural, non-laboratory style environments with non-skilled personnel conducting these processes.

The kits and system will allow farmers to break up tissue/cell samples to release nucleic acid from cell or viral particles, and extract nucleic acid and perform nucleic acid amplification procedures using the heating device. Said heating device will also contain a sensor to determine the change in optical properties, such as optical density, or fluorescence light emission, informing the farmer of the results. The disposables include a kit for breaking up cell(s)/tissue(s), a nucleic acid capturing device, amplification reaction chamber (e.g. in the form of an amplification reaction container) and various lyophilized reagents. The kit for breaking up cell(s)/tissue(s) comprises the grinding device and lysis reagents. The nucleic acid extraction kit is for purifying the nucleic acid from the lysed sample for transferring to the amplification reaction container.

The amplification reaction container allows for the amplification process to proceed and the results to be read by one or more optical sensor on the amplification device. The disposables are designed to be for single use only to prevent contamination in a field environment. The lyophilized (or freeze-dried) reagents allow for transport and storage of reagents to be undertaken without any cold chain. This lyophilization process will greatly reduce the cost of transport and storage, making it increasingly easy for users in rural areas to use this technology.

Accordingly, the described invention allows unskilled users to perform sample preparation, nucleic acid transfer, nucleic acid amplification and visualisation of amplification in a rural environment in the absence of laboratory equipment and laboratory skilled personnel.

1. A nucleic acid capturing device adapted for dipping into a liquid wherein a portion of the device contacting the liquid comprises a nucleic acid binding agent.

2. (canceled)

3. The nucleic acid capturing device according to claim 1, wherein the nucleic acid binding agent comprises silica (SiO₂), and wherein the portion of the device contacting the liquid comprises a surface functionalized with silica or comprises a porous structure comprising a nucleic acid binding agent (e.g. a silica membrane).

4. (canceled)

5. (canceled)

6. A method for capturing nucleic acid from a liquid comprising:
(i) providing a nucleic acid capturing device adapted for dipping into the liquid wherein a portion of the device contacting the liquid comprises a nucleic acid binding agent; and
(ii) contacting said portion of the device with the liquid,
wherein the nucleic acid binding agent comprises silica (SiO₂), and wherein the portion of the device contacting the liquid comprises a surface functionalized with silica or comprises a porous structure comprising a nucleic acid binding agent (e.g. a silica membrane).

7.-9. (canceled)

10. A grinding device comprising:
(i) a mortar comprising a base grinding portion and an upper portion wherein a first opening at the top of the upper portion is larger than a second opening at the base of the upper portion; wherein the second opening delineates the end of the top portion and opens to the base grinding portion; and
(ii) a pestle adapted to fit into the base grinding portion for grinding a material in the base grinding portion,
wherein the bottom of the base grinding portion has substantially the same dimension as the second opening, wherein the wall of the base grinding portion extends substantially perpendicularly from its bottom to the second opening of the top portion.

11. (canceled)

12. (canceled)

13. The grinding device according to claim 10, wherein the base grinding portion comprises a buffer and/or wherein the first opening is sealed with a removable seal.

14. (canceled)

15. A grinding method comprising grinding a material in a grinding device comprising:
(i) a mortar comprising a base grinding portion and an upper portion wherein a first opening at the top of the upper portion is larger than a second opening to the base grinding portion;
wherein the second opening delineates end of the top portion and opens to the base grinding portion; and
(ii) a pestle adapted to fit into the base grinding portion for grinding material in the base grinding portion.

16. A system for purification, analysis and/or detection of nucleic acid molecules comprising:
(i) the nucleic acid capturing device as defined in claim 1; and
(ii) a polymerase chain reaction (PCR) machine or a heating device, wherein the PCR or heating device is adapted to carry out nucleic acid amplification in a reaction container.

17. A system for extraction, purification, analysis and/or detection of nucleic acid molecules; comprising:
(i) the grinding device as defined in claim 10; and
(ii) the nucleic acid capturing device as defined in claim 1; and
(iii) a polymerase chain reaction (PCR) machine or a heating device, wherein the PCR or heating device is adapted to carry out nucleic acid amplification in a reaction container.

18. (canceled)

19. (canceled)

20. The system according to claim 16, further comprising a device for analysis, detection and/or identification of a target nucleic acid molecule.

21. The system according to claim 20, wherein the system further comprises at least one computer processor, wherein said computer processor is adapted to send a signal to stop and/or prevent access to the PCR machine and/or the heating device, and wherein the computer processor(s) communicate with other computer processor(s) in the system and/or with one or more other computer systems.

22. (canceled)

23. (canceled)

24. A method for extraction, purification, analysis and/or detection of nucleic acid molecules comprising:
(i) contacting the device as defined in claim 1 with a liquid (e.g. a binding buffer) to capture nucleic acid molecules;
(ii) optionally washing the portion of the device comprising a nucleic acid binding agent after step (i);
(iii) releasing the captured nucleic acid molecules from the device into a solution;
(iv) amplifying the nucleic acid molecules; and
(v) analyzing and/or detecting the nucleic acid molecules after step (iii) or (iv).

25. (canceled)

26. The method according to claim 24, wherein (iv) comprises amplifying the nucleic acid with PCR, nucleic acid sequence based amplification (NASBA), recombinase polymerase amplification (RPA), nicking enzyme amplification reaction (NEAR), rolling-circle amplification (RCA), helicase-dependent amplification (I), multiple displacement amplification (MDA), CRISPR/Cas based isothermal amplification, or isothermal amplification (e.g. mediated amplification (LAMP)).

27. The method according to claim 26, wherein (iv) comprises amplifying the nucleic acid molecules with PCR in a PCR machine or amplifying the nucleic acid molecules with isothermal amplification (e.g. LAMP) in a heating device.

28. (canceled)

29. The method according to claim 27, wherein the method further comprises at least one computer processor sending a signal to stop the PCR machine or heating device, allow access of the PCR machine or heating to an authorized person; and/or prevent access to the PCR machine or heating device.

30. (canceled)

31. The method according to claim 24, comprising grinding a material using a grinding device as defined in claim 10 prior to step (i).

32. (canceled)

33. A method for coating a portion of a device for capturing nucleic acid comprising:
(i) contacting a portion of the device with a solution of tetraethyloxysilane, tetramethoxysilane and/or vinyltriethoxysilane in ethanol for a time period;
(ii) adding multiple aliquots of an acid or a base to the solution periodically;
(iii) removing the portion of the device from the solution of (ii); optionally repeating (i) to (iii); and
(iv) optionally drying the device.

34. The method according to claim 33, further comprising repeating (i) to (iii) at least once, wherein each (i) comprises contacting a portion of the device with 10-90% v/v tetraethoxysilane, for the first instance and the first repeat,
wherein the time period for each (i) independently, may be or is approximately 20 minutes, for the first instance and the second repeat;
wherein the acid in (ii) comprises citric acid or hydrogen chloride;
wherein the base for each (ii) independently, may comprise or comprises approximately 17% w/v NaOH, for the first instance and the first repeat; and
wherein each (ii) of the first instance and the first repeat comprises adding 150 uL of 17% w/v NaOH solution at approximately 5 minute intervals.

35.-44. (canceled)

45. The method according to claim 33, wherein (i) to (iii) are repeated twice, wherein (i) for the second repeat comprises contacting a portion of the device with 10-90% v/v tetraethyloxysilane for the second repeat, wherein the time period for (i) for the second repeat independently, is approximately 30 minutes, wherein the concentration of sodium hydroxide solution for (ii) for the second repeat independently, is approximately 17% w/v.

46.-48. (canceled)

49. The method according to claim 45, wherein (ii) independently comprises adding 100 uL of 17% w/v NaOH solution at approximately 9 minute intervals.

50. The method according to claim 33, further comprising optionally sonicating a portion of the device in said solution after each (ii).
